(19) United States
(12) Patent Application Publication
(10) Pub. No.: US 2006/0173509 A1
(43) Pub. Date: Aug. 3, 2006

(54) Deep brain stimulator

(75) Inventors: Kendall H. Lee, West Lebanon, NH (US); David W. Roberts, Lyme, NH (US); Alexander Hartov, Lebanon, NH (US)

Correspondence Address: PALMER & DODGE, LLP;KATHLEEN M. WILLIAMS, 111 HUNTINGTON AVENUE, BOSTON, MA, 02199, US

(73) Assignee: Dartmouth College

(21) Appl. No.: 11/244,134

(22) Filed: Oct. 5, 2005

Related U.S. Application Data
(60) Provisional application No. 60/615,995, filed on Oct. 5, 2004.
(60) Provisional application No. 60/669,743, filed on Apr. 8, 2005.

(51) Int. Cl. A61N 1/32

(52) U.S. Cl. 607/45

(57) ABSTRACT

The present invention relates to a method for the detection and ablation of aberrant thalamic oscillations leading to tremor and/or seizure. The invention provides a high frequency stimulator useful for treating and/or preventing the onset of tremor and seizure.

### RELATED APPLICATIONS

This application claims priority under 35 U.S.C. § 119 U.S. Provisional Application Ser. No. 60/615,995 filed Oct. 5, 2004 and U.S. Provisional Application Ser. No. 60/669,743 filed Apr. 8, 2005, the entirety of which are incorporated herein by reference.

### BACKGROUND OF THE INVENTION

High frequency deep brain stimulation (HFS) applied to the thalamus is an effective treatment for epilepsy and drug resistant tremor. (Hodaie, et al., 2002, Epilepsia 43:603; Benabid, et al., 1996, J. Neurosurg. 84:203; Coller, et al., 2000, Neurology, 55:S29; Lozano, 2000, Arch. Med. Res. 31:266.) Intraoperative recordings from patients have shown that tremor neurons in the thalamus discharge rhythmically either prior to, or in synchronization with, the 3 to 6 Hz oscillatory muscular tremor. Similarly, absence-like seizures are associated with 3 Hz spike and wave oscillations in the EEG and probably represent a perverse form of thalamocortical activity that is related to the normal generation of spindle waives. (McCormick and Contreras, 2001, Annual Review of Physiology, 63: 815.) There is a need, however, for a mechanism to regulate and prevent the onset of epilepsy and tremor in an individual, e.g., based on the detection of specific electrical and/or chemical events in the thalamus. There is also a need for a means to stop tremor onset prior to reaching the acute stage; that is, prior to the physical manifestation of tremor.

### SUMMARY OF THE INVENTION

The present invention provides high frequency stimulation to the brain of an individual in response to detection of an epileptic oscillation using a deep brain stimulation/monitoring system. The high frequency stimulation system includes a control module, a detection module having electronic circuitry capable of detecting an epileptic oscillation and a high frequency stimulation module under control of the control module. The detection module, which may be part of the control module, monitors for the presence of an epileptic oscillation. If the epileptic oscillation is present, a signal is sent to the control module, which in turn notifies high frequency stimulation module to generate high frequency stimulation to the brain under the control of the control module.

The invention also relates to a method for reducing tremor in an individual. A sensor capable of measuring an epileptic oscillation in the individual and operatively connected to a control module is placed in or on the brain of the individual. A stimulation electrode, operably connected to the control module, is placed in or on the brain of the individual. Thalamic oscillation is measured from the individual, and an epileptic oscillation, if present, is detected in the thalamic oscillation. Upon detection of an epileptic oscillation, a signal indicative thereof is sent from the control module to the high frequency stimulation module. The high frequency stimulating module generates a high frequency stimulation signal under control of the control module. The tremor is reduced by the high frequency stimulation signal applied to the stimulation electrode. The stimulation electrode may be placed in or in proximity to the thalamus of the individual.

The epileptic oscillation detected according to the invention is a thalamic oscillation of 3 to 6 Hz, but may also be evidenced as a decrease or change in thalamic oscillation from 7-14 Hz.

The high frequency stimulation system optionally includes a chemical delivery module operatively connected to the control module. The high frequency stimulation method may, thus, include delivery of a neuroactive compound by the chemical delivery module in response to a signal from the control module.

Neuroactive compounds useful in the invention includes, but is not limited to neurotransmitters, neuropeptides, neurochemicals, receptor agonists, receptor antagonists, ion channel blockers, ion channel activators, and calcium chelators.

One of skill in the art will readily appreciate that one can measure thalamic oscillation in myriad ways, including, but not limited to EEG recordings.

The present invention further features a system for treating tremor in an individual. The system includes a control module, a high frequency stimulating module, a sensor adapted to be placed in or on the brain and capable of recording a thalamic oscillation signal, and a stimulating electrode. The high frequency stimulating module is operatively connected to the control module, as is the sensor. The stimulating electrode is connected to the high frequency stimulating module. The control module includes electronic circuitry that determines if an epileptic oscillation is present in the thalamic oscillation based on input from the sensor and sends a signal to the high frequency stimulating module if the epileptic oscillation is present. The high frequency stimulation module then generates a high frequency stimulation that is delivered to the brain via the stimulating electrode.

Accordingly, the present invention provides a method for maintaining normal thalamic oscillations by sensing the onset of seizure-like activity and modulating transmitter levels accordingly via high frequency stimulation.

### BRIEF DESCRIPTION OF THE FIGURES

FIG. 1 shows a flowchart of a feedback circuit of the present invention which may be used to maintain normal thalamic oscillatory activity.

FIG. 2 shows a block diagram of a high frequency stimulator useful in the present invention.

FIG. 3 shows a more detailed block diagram of the high frequency stimulator of the invention which shows additional components that may be included in the high frequency stimulator.

FIG. 4 shows a block diagram representation of the high frequency stimulator in which a chemical delivery module is included for the delivery of neuroactive compounds directly to the brain.

FIG. 5 shows a block diagram of a high frequency stimulator in which both the sensor and stimulating electrodes are combined.

FIG. 6 shows intracellular and extracellular recordings of thalamic neurons demonstrating spontaneous spindle wave generation.

FIG. 7 shows simultaneous recordings of spindle oscillations made with an extracellular and an intracellular electrode in lamina A1 of the ferret LGN.

FIG. 8 shows extracellular recordings from lamina A1 of the ferret LGN slice with GABA_{A} antagonist picrotoxin (20 μM) in bath.

FIG. 9 shows intracellular current clamp recording from a thalamocortical relay neuron in the presence of the GABA_{A} antagonist picrotoxin.

FIG. 10 shows a more detailed example of a deep brain stimulator of the invention.

FIG. 11 is a schematic drawing showing an example of a single probe comprising electrical recording and stimulation electrodes.

FIG. 12 shows subthalamic nucleus (STTN) and ventrolateral (VL) thalamic glutamate release with high frequency stimulation (HFS) in the rat in vivo.

FIG. 13 shows HFS in the ferret thalamic slice results in glutamate release that is not blocked by the classic neuronal exocytosis inhibitors, TTX or low Ca++, high Mg++ bath solution.

FIG. 14 shows GFAP staining and Glutamate release in primary astrocytic cultures.

### DETAILED DESCRIPTION

The present invention provides a method and apparatus for the detection and monitoring of epileptic oscillations in the thalamus of an individual. The invention features a mechanism for detecting the onset of an epileptic event and provides high frequency stimulation of the brain in response to treat the condition. More particularly, high frequency stimulation of the thalamus is used to eliminate and/or prevent the onset of tremor or seizure activity.

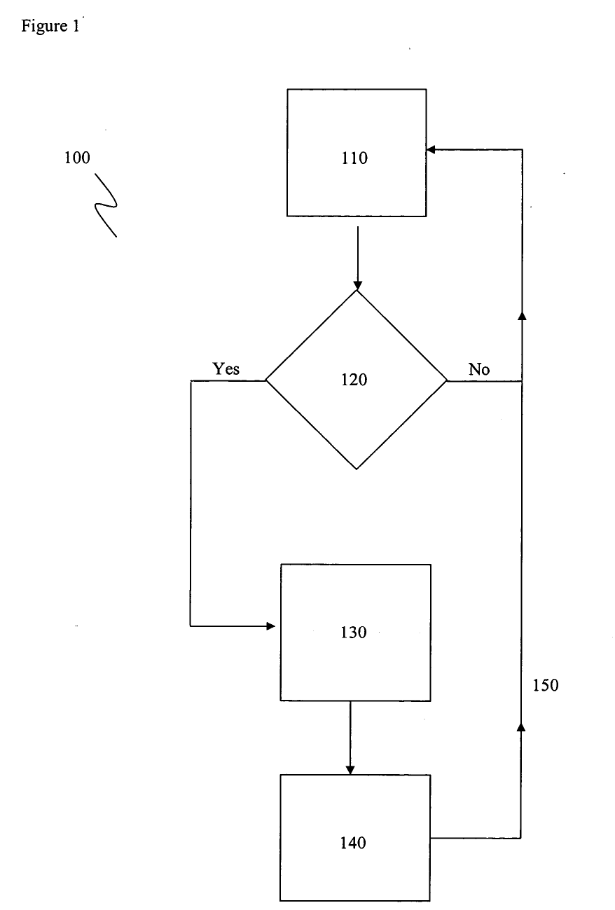

FIG. 1 is a flow chart illustrating general feedback loop 100, which is used to treat or prevent seizure and/or tremor in an individual. In first step 110, brain activity is monitored to measure and detect thalamic oscillations. It is well understood that the thalamus of most normal individuals has a baseline oscillatory frequency of between 7 and 14 Hz. Thalamic oscillations may be monitored by use of electroencephalogram (EEG) recordings. Other methods that can be used to monitor thalamic activity and detect epileptic oscillations include extracellular tungsten microelectrodes (Frederick Haer Company, Bowdoinham, Me.), and extracellular macroelectrode depth electrode recording. Alternatively, one of skill in the art can measure thalamic oscillation by monitoring changes in glutamate levels in the brain using, for example, constant potential amperometry (for example, glutamate sensors are being developed by Pinnacle Systems, Mountain View, Calif.). Since glutamate is one of the primary neurotransmitters used by thalamus, monitoring changes in glutamate levels thalamus is indicative of the electrical activity in thalamic neurons as well as thalamocortical relay neurons. Methods for detecting neurotransmitter levels using constant potential amperometry are known (see, e.g., Blaha, et al., 1996, J. Neurosci. 16: 714; Blaha and Lane, 1984, Euro. J. Pharmacol., 98: 113; Blaha and Phillips, 1990, J. Neurosci. Methods, 34: 125; and Bergman, et al., 1990, Science, 249: 1436).

Thalamic oscillations may be measured directly from the thalamus or specific nuclei thereof. Alternatively (or in addition), thalamic oscillations may be measured from other brain regions in communication with the thalamus, as well as afferent and/or efferent fiber tracts of the thalamus, or thalamocortical pathways.

In second step 120, the thalamic oscillations are examined to determine whether an epileptic oscillation is present. As used herein, an “epileptic oscillation” refers to electrical oscillation in the thalamus of an individual having a frequency of between 3 and 6 Hz. Normal sleep spindle thalamic oscillatory activity is in the range of between 7 and 14 Hz. Epileptic events have been shown to be associated with 3-6 Hz thalamic oscillation for tremor and 3 Hz oscillation for absence-like seizure. “Epileptic oscillation” also refers to a measurement or detection of a change in the thalamic oscillation frequency from about 7-14 Hz; for example, a decrease in thalamic oscillation from about 7-14 Hz to about 3-6 Hz. An epileptic oscillation may be detected by oscillations in neurotransmitter levels that are indicative of electrical oscillation in the thalamus. For example, epileptic oscillations may be detected as oscillating levels of glutamate in the thalamus, which is indicative of electrical oscillation.

Step 120 of detecting epileptic oscillation may be performed using hardware, software, or firmware. For example, the detection of epileptic oscillation may be performed using hardware such as a gating or filtering circuit that only permits the transmission of signals detected in step 110 having the characteristics of an epileptic oscillation. Such circuits are well known in the art. Alternatively, step 120 may utilize a processor programmed with firmware or software to analyze the thalamic oscillations detected in step 110 to identify the onset of an epileptic oscillation. If no epileptic oscillation is detected in step 120, then step 110 and 120, are repeated. If an epileptic oscillation is detected in step 120, then, in step 130, a signal is sent from the processor used in step 120 to a high frequency stimulation module which is capable of delivering electrical stimulation to the brain of the individual. The signal may be sent directly, or indirectly. For example, the signal may also be relayed so that a neuroactive compound is administered before, after, or coincident with the electrical stimulation provided by the high frequency stimulator. In step 130, the high frequency stimulation module generates an electrical signal having the parameters outlined in Table 1. The specific stimulation parameters may be modified by one of skill in the art to meet a particular application without departing from the scope of spirit of the invention.

TABLE 1
High Frequency Stimulation Parameters

| Amplitude | Output Voltage | Pulse Width | Frequency | Duration |
|---|---|---|---|---|
| 10-1,000 μA | 1-20 V | 10-15 μs | 10-500 H_{z} | 1 sec-12 hours |

In step 140, the high frequency electrical stimulation generated in step 130 is applied to the brain of the individual to be treated. The high frequency electrical stimulation may be applied directly to the thalamus or to specific nuclei or cell populations thereof. Alternatively, or in addition, the stimulation may be applied to afferent or efferent thalamic fiber tracts, or other cortical or subcortical regions that are interconnected with the thalamus. In step 150, a signal is also sent back to step 110 to reset the system and start further measurements of thalamic oscillation.

The method shown in FIG. 1 may be carried out using any of the high frequency stimulation systems described herein. Modification of the above described method to conform to particular aspects of an individual are within the scope of the invention, and the method may be readily adapted by one of skill in the art.

The present invention also relates to a system for providing high frequency stimulation to the brain, or specific brain regions, of an individual in response to the detection of particular electrical oscillation in the thalamus, or structures interconnected thereto.

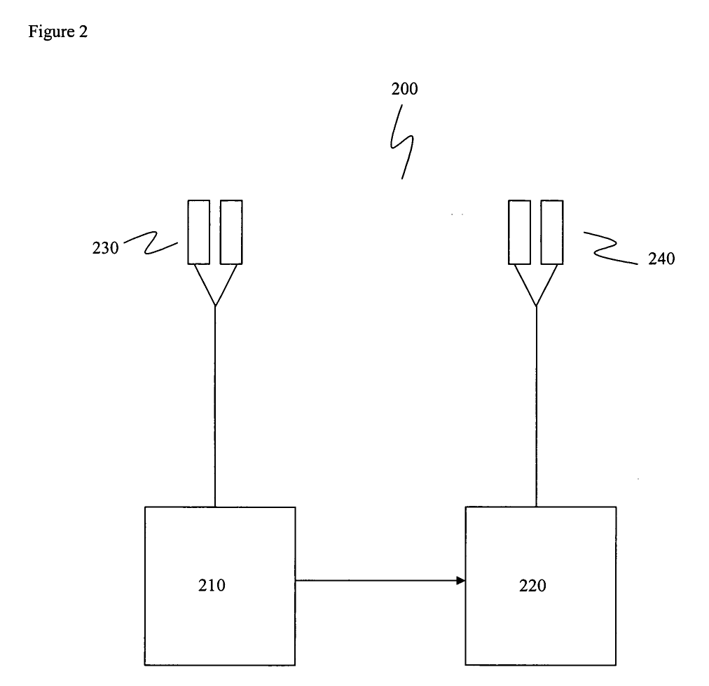

FIG. 2 shows a high frequency simulator 200 of the invention. Stimulator 200 comprises minimally, sensor and stimulation electrodes 230 and 240, respectively, control module 210, and high frequency stimulation module 220. One of skill in the art will appreciate that the electrodes 230 and 240, instead of being separate and distinct electrodes as shown in FIG. 2, may each be a single electrode or a single plurality of electrodes which perform the function of both of electrodes 230 and 240. For example, electrodes 230 and 240 may be included on a single implantable probe. Electrodes 230 and 240 are embedded within or proximate to the brain of an individual to be monitored. Sensor electrode 230 may be any sensor that is suitable for measuring or sampling brain wave activity in an individual. Sensor electrode 230 may be a single electrode or a plurality of electrodes, including removable electrodes that are placed on the scalp of an individual. Electrode 230 is of a type known in the art and removable electrodes are available from commercial sources such as Grass-Telefactor (West Warwick, R.I.). Alternatively, sensor electrode 230 can be a single needle, or a plurality of needles, which are capable of recording electrical activity (e.g., EEG) in a specific brain region into which they are placed. Additional examples of sensor electrodes 230 include an extracellular tungsten microelectrode and an extracellular macroelectrode depth electrode. Sensor electrode 230 may be adapted for temporary or permanent placement in the brain of an individual to permit continuous sampling of brain wave activity of the individual. Alternatively, sensor electrode 230 may comprise a constant potential amperometer, which is capable of detecting glutamate release from, for example, the thalamus. Methods and sensors for the detection of neurotransmitters by amperometry are known in the art.

Control module 210 includes electronic circuitry that is adapted to receive signals from sensor electrode 230, determine whether an epileptic oscillation is present, and to send a signal to the high frequency stimulation module 220. The circuitry may include a gating or filter circuit, which is designed to only allow electrical signals having set properties trigger a control signal to, for example, the high frequency stimulation module 220. Circuits of this type are known in the art and may be readily adapted for use in the instant invention. Alternatively, control module 210 may comprise other hardware, firmware, or software designed to detect an epileptic oscillation in the thalamic oscillation signals provided by sensor electrode 230. Control module 210 is operably connected to high frequency stimulation module 220, which, in turn, is capable of generating electrical signals having the properties shown in Table 1. Preferably, high frequency stimulation module 220 generates an electrical signal at 100 Hz or greater. High frequency stimulation module 220 is operably connected to stimulation electrode 240 such that an electrical signal generated by high frequency stimulation module 220 is conveyed to stimulation electrode 240 and thus directed into and/or onto the brain of the individual being treated. Stimulation electrode 240 may be any conductive electrode that is capable of delivering an electrical stimulus to brain tissue of an individual. Stimulation electrode 240 includes surface electrodes which may be removably placed on the scalp of the individual, and/or coaxial or other suitable electrodes which are placed directly in the brain of an individual to be treated. While the electrodes 230 and 240 generally must be located in or on the individual to be treated (particularly, in or on the brain of the individual), the other components of high frequency stimulator 200 may be located externally. For example, control module 210 and high frequency stimulation module 220 may be removably attached to the individual (e.g., by a belt clip, harness, lanyard), or alternatively, may be miniaturized to suitable size for implantation in an individual, e.g., implanted under the skin in the abdomen, chest, or neck. Control module 210 and high frequency stimulation module 220 may be connected to each other and to the sensor and stimulation electrodes 230 and 240 by suitable means known to those of skill in the art. These include, but not limited to wire, coaxial cable, optical cable, fiber optics, or infrared signals. Control module 210 and high frequency stimulation module 220 may be remote from one another, or control module 210 and high frequency stimulation module 220 may be incorporated into the same device by way of a housing, case, shell, frame, or other suitable mechanism, or packaging. One or more elements of the high frequency stimulator 200 may be permanently connected, for example, control module 210 and high frequency stimulation module 220 may be contained within a housing or other confinement and permanently connected by solder or other electrically conductive weld.

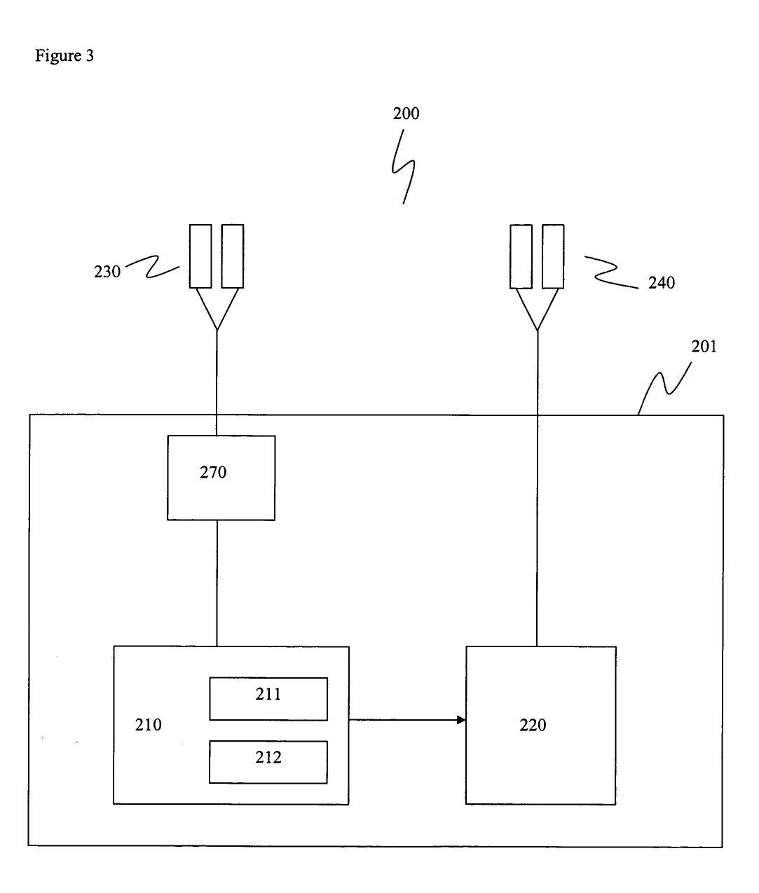

FIG. 3 shows a more detailed schematic of high frequency stimulator 200. As shown in FIG. 3, additional components such as an amplification and conversion device 270 and a detection signal processor 211 may be included. Amplification and conversion device 270 may be interposed between sensor electrode 230 and control module 210. There are a number of commercial vendors who provide devices suitable for measurement of thalamic oscillations. In general, amplification and conversion device 270 should be capable of at least >20 ms waveform sampling, have at least 4 channel inputs for electrodes 230 and 240 (and can have up to 16, 32, and 128 inputs), and analog or digital inputs and outputs. Amplification and conversion device 270 should be able to interface with other possible components of the high frequency stimulation device 200, including control module 210, and detection signal processor 211. In particular, amplification and conversion module 270 should have an independently adjustable gain for each channel that is adjustable across a small range such as a maximum of 200,000 and a minimum of 50. Amplification and conversion device 270 may include also band-pass filtering from 0.11 Hz to 16 Hz (although the high cutoff frequency filtering may be as high as 100 Hz).

For example, an amplifier and conversion device 270 of the present invention will comprise an amplifier which has specifications, examples of which are shown in Table 2:

TABLE 2
Amplifier Specifications

| Parameter | Value |
|---|---|
| Input Impedence | >200 MΩ/25 pF; |
| Sensitivity | 1 V/20 μV-1 V/10 mV |
| High Frequency filter | 100 Hz to 15 Hz in 8 steps, 6 dB/octave |
| Low Frequency filter | 0.5 Hz to 500 Hz in 8 steps, 6 dB/octave |
| Notch filter | >30 dB down at 60 Hz |
| CMRR | >100 dB at 60 Hz |
| Noise | <1 micro Volt rms from 2 Hz-10 kHz with |
| | input shortened |
| Calibration | 100 Hz squarewave, 2 μV/div to 10 mV/div |
| | in 12 steps |
| Temperature measurement | 20° C.-40° C. |

The amplification and conversion device 270, in addition to being capable of amplifying an electrical signal, may be able to convert an analog electrical signal to a digital signal for transmission of the signal to the control module 210, described further below. Amplification and conversion device 270 may also be capable of converting a digital signal to an analog signal. Methods and mechanisms for the conversion of analog to digital and digital to analog are well known to those of skill in the art and may be readily incorporated into an amplification and conversion device 270. The amplification and conversion portions of the amplification and conversion device 270 may be included in a single unit or component, or may be separate components of the high frequency stimulator (i.e., physically separable components of the simulator).

Control module 210 may include a detection signal processor 211, which is capable of performing the processing of step 120 shown in FIG. 1. Thus, detection signal processor 211 may comprise hardware, such as a gating or filter circuits, or may have a processor analogous to a general purpose computer programmed with firmware or software adapted to perform the detection step 120 of FIG. 1. Parameters for detection of epileptic oscillation by detection signal processor 211, which include certain characteristics of detected electrical signals that indicate the onset of tremor, may be programmed into parameters 212. Detection parameters 212 may be permanently set, or may be adjustable by either the individual, or by a physician treating the individual. Any data processed by detection signal processor 211 or created as a result of such processing may be optionally stored as memory as is conventional in the art. For example, such data may be stored in a temporary memory such as in the RAM of a given computer system or subsystem. In addition, or in the alternative, such data may be stored in longer-term storage devices, for example, magnetic disks, rewritable optical disks, and the like. For purposes of the disclosure herein, a computer-readable media may comprise any form of data storage mechanism, including such existing memory technologies as well as hardware or circuit representations of such structures and of such data.

In addition to the components described above, control module 210 may further comprise conventional peripherals, including input devices and output devices, such as a LCD display, speaker, vibration generator, light, or other output device which may be used to communicate the detection of an epileptic oscillation. Control module 210 may be a computer, such as a PC, or may be connected to a computer. A computer can be a standard personal computer, or may be adapted from, for example, a handheld computing device such as a PDA or SNAP module from Nicolet Biomedical (which includes EEG recording capabilities).

Detection of an epileptic oscillation by control module 210, or more specifically, by detection signal processor 211, if present, triggers a signal to be sent from control module 210 to high frequency stimulation module 220. High frequency stimulation module 220 then provides a high frequency stimulation via electrodes 240 to the brain of the individual in which the epileptic oscillation was detected.

It will be appreciated by one of skill in the art that electrical isolation may be provided between components of the high frequency brain stimulator. For example, electrical isolation may be provided between stimulation module 220 and control module 210, and or between the control module 210 and amplification and conversion device 270. Electrical isolation may be achieved using methods or components known in the art such as optical isolation.

The components of stimulator 200 may be arranged such that they are remote from one another. The components of stimulator 200 are commonly operably connected by means of wire, coaxial cable, optical cable, fiber optics, or infrared signals. Alternatively, several or all of the components of stimulator 200 may be in close spatial proximity such that they are operably connected by solder, other electrically conductive weld, or as part of a printable circuit. The components of stimulator 200 may be incorporated into a single device 201 by way of a housing, case, shell, frame, or other suitable mechanism, packaging, or confinement known to those of skill in the art. Packaged device 201 may be worn externally, such as on a belt-clip, harness, or lanyard, or may be implanted, such as under the skin of the chest, back, neck, or abdomen. Device 201 or components thereof are connected to electrodes 230 and 240 in the brain by means of wire, coaxial cable, or optical cable.

The present invention is based, in part, on the discovery that application of high frequency stimulation to the brain of an individual displaying epileptic oscillations abolishes tremor and seizure-like activity, and triggers the release of neurotransmitters from the thalamus. In particular, HFS has been shown to stimulate the release of glutamate from the thalamus. Without being bound to one particular theory, it is believed that glutamate release may be the underlying mediator of HFS induced abolition of tremor and seizure. Accordingly, the invention can include, in addition to the high frequency stimulation system taught herein, a chemical delivery system for administering neuroactive compounds (e.g., glutamate) in response to epileptic oscillations.

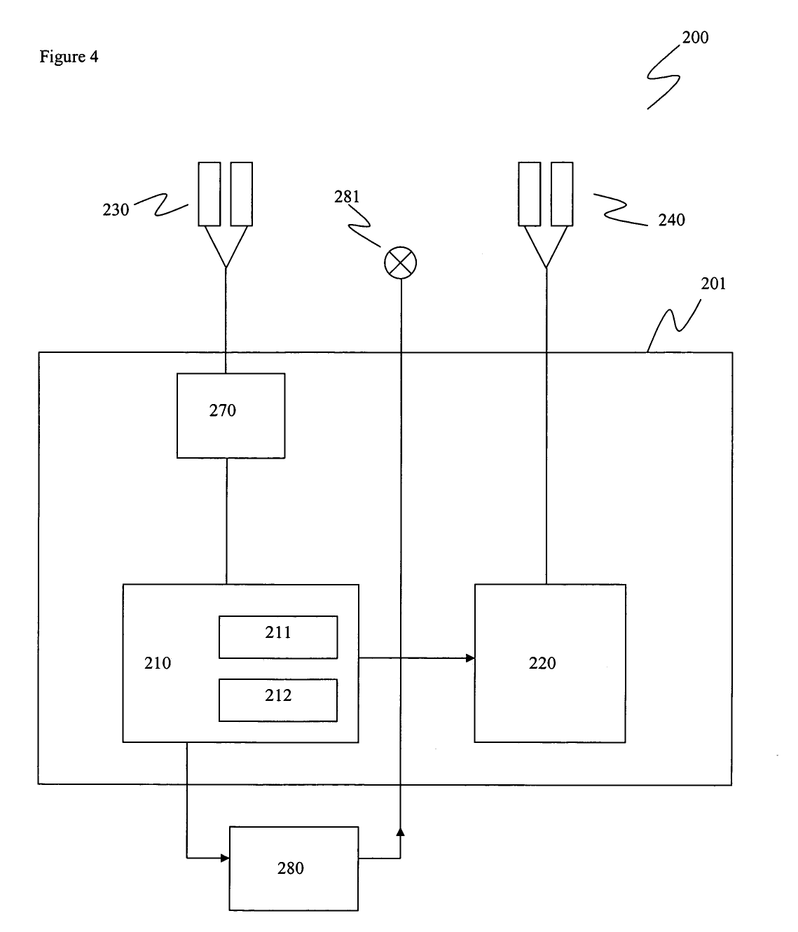

FIG. 4 shows a further embodiment of the high frequency stimulator 200 of the present invention. Stimulator 200, shown in FIG. 4, includes a chemical delivery module 280 operably connected to control module 210. Upon detection of an epileptic oscillation, control module 210 may also send a signal to chemical delivery module 280 in addition to sending a signal to high frequency stimulation module 220. Control module 210 may be programmed to trigger the release of neuroactive compounds using different patterns. In one such pattern, each time control module 210 sends a signal to high frequency stimulation module 220 to generate electrical stimulation, a signal is also sent to chemical delivery module 280, causing it to release neuroactive compound. Alternatively, release of neuroactive compound from chemical delivery module 280 may be regulated by control module 210 based on a particular dosing regimen prescribed by a physician. For example, detection of an epileptic oscillation by control module 210 will only send 1, 2, 3, or 4 or more signals to chemical delivery module 280 in a given period (e.g., every 24, 48 or 72 hours, or one dose every 6, 12, or 24 hours). Chemical delivery module 280 preferably comprises a reservoir, capable of containing a neuroactive compound, and a pump, or its equivalent. Upon receipt of an appropriate signal, chemical delivery module 280 delivers the chemical (i.e., via a pump) from the reservoir to delivery module 281. Delivery module 281 may be a needle, syringe, catheter or other tubing, which is implanted or removably placed in close proximity to the site at which delivery of the chemical is desired (e.g., the brain, more specifically, the thalamus). The pump of chemical delivery module 280 may be a peristaltic-type pump or a mini-osmotic-type pump, such as those available from Alzet (Cupertino, Calif.). The chemical delivery module 280 may be incorporated in a housing 201 that also includes control module 210, amplification and conversion device 270 and high frequency stimulation module 220. Alternatively, chemical delivery module may be remote from the other components of the high frequency stimulator 200. The high frequency stimulator may be contained in a housing 201 that is implanted in an individual or worn externally. In addition, chemical delivery module 280 can be implanted in the individual separately from housing 201. For example chemical delivery module 280 may be implanted in the abdomen or under the skin of the chest, wherein a tube or catheter extends from chemical delivery module 280 to delivery module 281 which is on, in, or near the thalamus of the individual. Alternatively, all the components of high frequency stimulator 200, including chemical delivery module 280, are worn externally.

As indicated above, the chemical delivery module 280 can be operably connected to control module 210, such that a signal from control module 210 triggers release of chemical from chemical delivery module 280 via delivery module 281. Alternatively, chemical delivery module 280 can be manually controlled by the individual using a switch or other device. In this mode, detection of an epileptic oscillation by control module 210 triggers some output means which may be perceived by the individual. For example, the control module 210 may issue a tone, light, vibration, or mild electronic shock to signal the detection of an epileptic oscillation. After perceiving the signal produced by control module 210, the individual can choose whether to manually trigger the chemical delivery module such that neuroactive chemical is delivered to the brain of the individual.

Chemical delivery module 280 may be used to deliver to an individual any composition of interest, e.g., a neuroactive compound. Preferably, the neuroactive compound is chosen from the group of neurotransmitters, neuropeptides, neurochemicals, receptor agonists, receptor antagonists, ion channel blockers, ion channel activators, and calcium chelators. Neuroactive compounds selected from gluatmate, GABA, serotonin, norepinepherine, and dopamine are preferred. Other neuroactive compounds are contemplated by the invention and may be included in chemical delivery module 280 as desired by one of ordinary skill in the art.

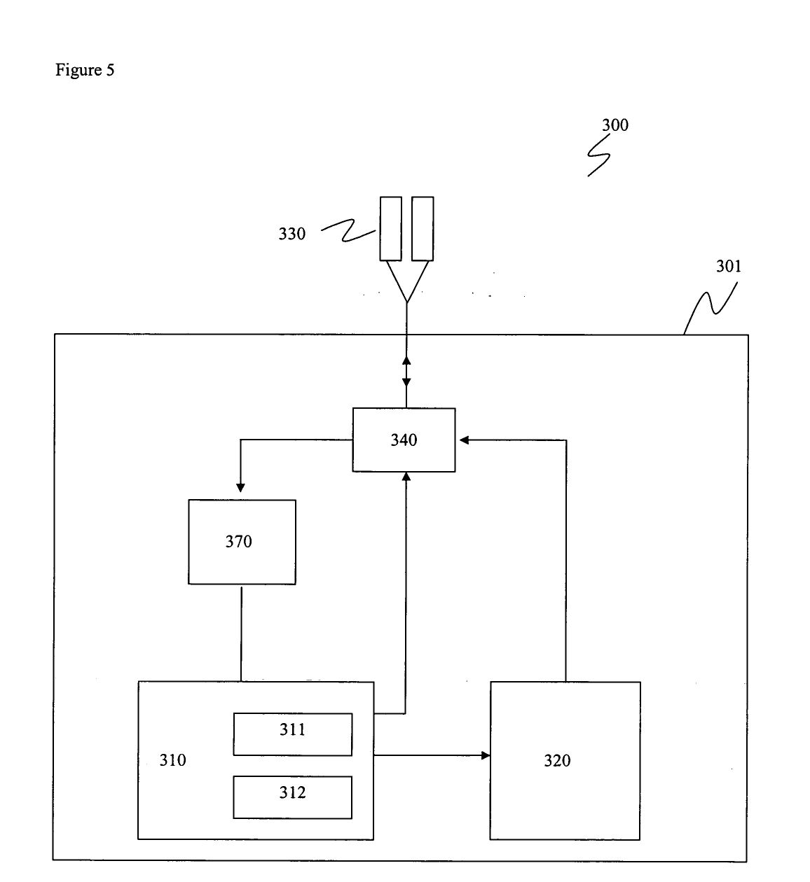

FIG. 5 shows a high frequency stimulator 300, which is adapted from high frequency stimulator 200 to include a combined sensor and stimulation electrode 330. It will be appreciated by one of ordinary skill in the art that electrodes 230 and 240 of stimulator 200 may be combined to a single electrode (or a single plurality of electrodes) such as electrode 330. Stimulator 300 also optionally includes a switch 340, which may be controlled by control module 310, to alternate between detecting thalamic oscillation and delivering high frequency stimulation. Electrode 330 may be a single electrode of any of the types discussed hereinabove, or may be a plurality of electrodes 330, the signals to and from which coalesce on switch 340. Alternatively, electrode 330 can include both a detection electrode and stimulation electrode on a single implantable probe. It will be understood by one of skill in the art that high frequency stimulator 300 may be adapted, similar to stimulator 200 shown in FIG. 4, to include a chemical delivery module 280.

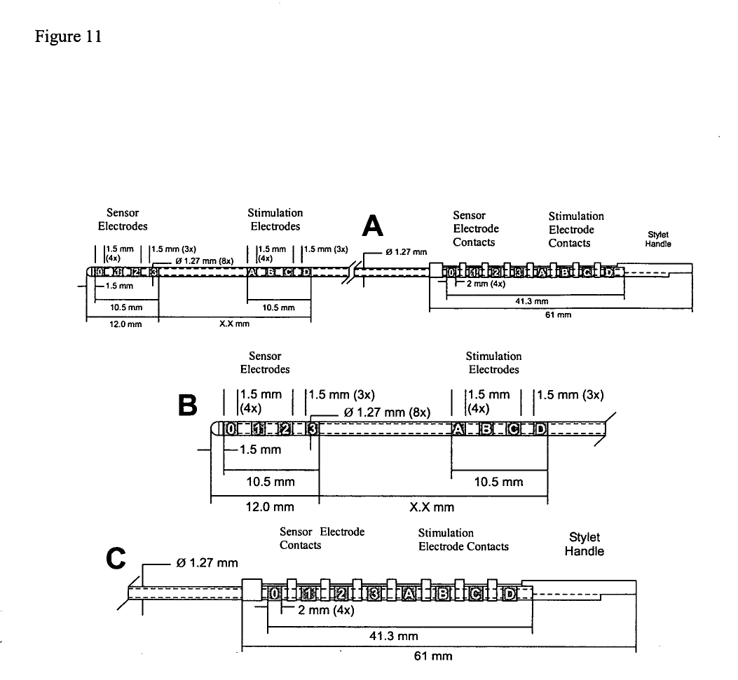

The stimulation electrode and sensor electrode may be further adapted to be included on a single probe for implantation in the brain of an individual. FIG. 11 shows a depiction of a combined sensor and deep brain stimulation electrodes on a single probe. FIG. 11A is a full view of the probe showing the sensor and stimulation electrodes as well as the stimulation electrode contacts and sensor contacts (i.e., where connection is made to the other components of the deep brain stimulator of the invention). Stimulation electrodes labeled 0-3 comprise four individual platinum-iridium ring electrodes for electrical stimulation of brain tissue. Although FIG. 11A shows four stimulation electrodes, the number of stimulation electrodes may be as few as one, or more than four. It will be appreciated by one of skill in the art this embodiment of the invention is not limited to the use of platinum-iridium for the stimulation electrodes, but that other conductive materials may be used within the scope of the invention.

As shown, sensor electrodes labeled A-D comprise four individual platinum-iridium ring electrodes for detection of epileptic oscillation. Although FIG. 11A shows four sensor electrodes, the number of sensor electrodes may be as few as one, or more than four. One of these electrodes may serve as an auxiliary/reference electrode. Electrode contacts labeled 0-3 and A-D, respectively, permit individual electrical contact with the high frequency brain stimulator of the invention. Although FIG. 11 shows the same number of sensor and stimulation electrodes on a given probe, it will be understood by one of skill in the art that the respective numbers of sensor and stimulation electrodes may vary relative to one another. The stylet handle permits permanent connection of the probe with a chronically implanted high frequency brain stimulator. The distances between components of the combined probe shown in FIG. 11 are for example only, and may be modified as needed for a particular individual or application. The distance X.X mm separating the sensor and stimulation electrodes on the shaft of the probe is a variable distance, and will ultimately correspond to the specific dorsal-ventral or medial-lateral distance separating the brain structures to be stimulated and recorded. FIGS. 11B and 11C are depictions of the same probe shown in FIG. 11A, but expanded in size for clarity of the component parts of the probe.

Modification of the particular processing performed by the high frequency stimulation device 200 and/or 300, or modify the particular components of the device 200 and/or 300 to fit the needs of a particular individual or circumstance, is within the scope of the present invention.

### EXAMPLES

### Example 1

The following experiments utilized ferret thalamic slices, which maintain an intact neural network and manifest spontaneous network oscillations, to examine the intracellular effects of HFS on thalamic neurons. The experiments test the hypothesis that HFS abolishes synchronized oscillations, such as spindle waves and 3 Hz absence-like seizure-like discharges, by releasing neurotransmitters.

Methods

Slice Preparation:

For the preparation of slices, male or female ferrets (Mustela putorious furo; Marshall Farms; North Rose, N.Y.), 2-4 months old, were deeply anesthetized with sodium pentobarbital (30-40 mg/kg) and killed by decapitation. The forebrain was rapidly removed, and the hemispheres were separated with a midline incision. Four hundred micron thick slices were cut using a vibratome (Leica Microsystems, Nussloch, Germany) in the sagittal plane. A modification of the technique developed by Aghajanian and Rasmussen (1989 Synapse 3:331) was used to increase tissue viability. During preparation of the slices, the tissue was placed in a solution (˜5° C.) in which NaCl was replaced with sucrose while maintaining an osmolarity of ˜307 mOsm. After preparation, the slices were placed in an interface style recording chamber (Fine Sciences Tools), maintained at 36±1° C. and allowed to recover for at least two hours. The bathing medium contained: 126 mM NaCl, 2.5 mM KCl, 1.2 mM MgSO₄, 1.25 mM NaH₂PO₄, 2 mM CaCl₂, 25 mM NaHCO₃, 10 mM dextrose, and was equilibrated with 95% O₂, 5% CO₂ to a final pH of 7.4. For the first 20 minutes, the thalamic slices were placed in the recording chamber and perfused with an equal mixture of the normal NaCl and the sucrose-substituted solutions. Subsequently, the slices were perfused only with the normal NaCl solution.

Electrophysiology

Intracellular recording electrodes were formed on a Sutter Instruments P-87 micropipette puller from medium-walled borosilicate capillaries (1B100F, WPI, Sarasota, Fla.). Micro-pipettes were filled with 2 M K-acetate and had resistances of 60-100 MΩ. Only those neurons exhibiting a stable resting membrane potential of less than −55 mV were included for analysis. A concentric stimulating electrode was connected to an Iso-flex current isolator (AMPI, Jerusalem, Israel) and Master 8 stimulator (AMPI, Jerusalem, Israel) to deliver the stimulation (parameters: 10-1000 μA amplitude; 100 μs pulse width; 100 Hz frequency; 1-60 seconds). The tip of the stimulating electrode was placed in the A1 lamina of the LGN. The data was analyzed using eDAQ Chart (eDAQ Pty Ltd, Denistone East, Australia) on a Pentium style computer. Figures were drawn using CorelDRAW (Corel, Ontario, Canada),

Results

Spindle Wave Generation

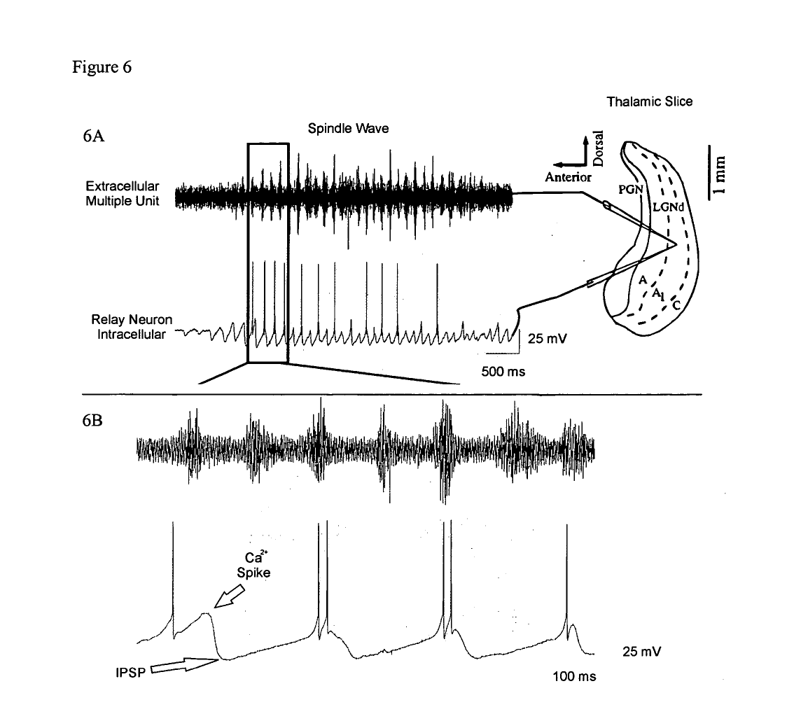

Simultaneous extracellular and intracellular recordings were obtained from the thalamocortical relay neurons in lamina A1 of the dorsal lateral geniculate nucleus (LGNd) in ferret thalamic slices maintained in vitro that showed spontaneous spindle wave generation (n=21 slices—see FIG. 6A). Spindle oscillations have been described as 1-3 second epochs of synchronized 7-14 Hz oscillations that are generated as a result of interactions between thalamocortical relay and thalamic reticular/perigeniculate neurons. (Bal et al., 1995 J. Physiol 483: 665; Bal et al., 1995 J. Physiol 483: 641) During the occurrence of spindle waves, intracellular recordings from LGNd thalamocortical relay neurons received barrages of IPSPs at a frequency of 7-14 Hz and these IPSPs resulted in the generation of rebound low threshold Ca²⁺ spikes (see FIG. 6).

High Frequency Stimulation During Spindle Wave

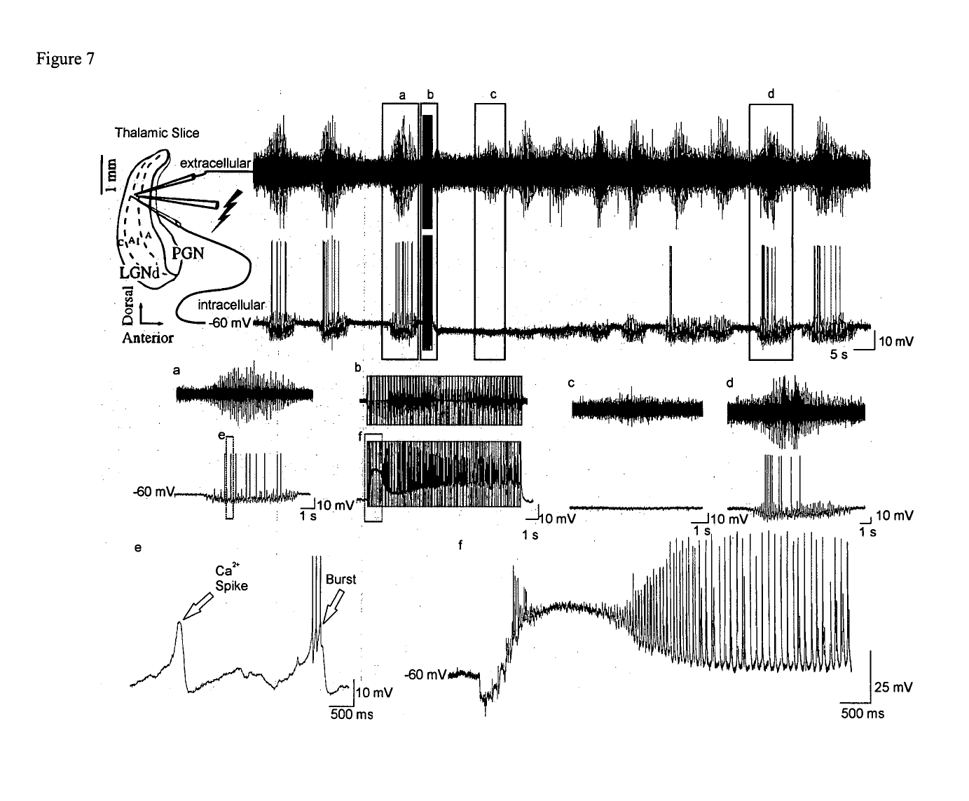

Spindle activity was recorded from a population of neurons using an extracellular electrode, and the electrophysiological activity associated with the spindle activity was recorded from single neurons within that population using an intracellular electrode (see FIG. 7). The intracellular recording (FIG. 7, frame a) revealed synchronized Ca²⁺ bursts (lower trace) concurrently with the population spindles (upper trace). The other traces shown in FIG. 7 correspond to time expanded views of the frames indicated as (b)-(f). HFS was applied by a stimulating electrode positioned within ˜100 μm of the intracellular and extracellular recording electrodes (n=12 slices). During HFS, it was not possible to observe the effect of HFS on spindle activity during the stimulation period due to the stimulation artifact in the extracellular trace. However, intracellularly, the stimulation artifact did not prevent the observation of an initial IPSP followed by a prolonged EPSP, membrane depolarization, action potential generation, depolarization block and further action potential generation (see FIG. 7, frame (f)). More specifically, FIG. 7, frame (f) shows enlargement of a section of the intracellular recording in frame b with the stimulation artifacts manually removed using CorelDRAW. An initial IPSP followed by several EPSPs, action potential generation, depolarization block, and further generation of action potential activity can be seen. The presence of IPSPs and EPSPs suggest that HFS results in synaptic neurotransmitter release. In the immediate post-stimulation period, neuronal activity was absent. The activity returned gradually in approximately 10-30 seconds while spindling returned in 30-60 seconds (FIG. 7, frame (d)), indicating that the neurons were not lesioned or damaged.

High Frequency Stimulation During Slowed Oscillations

Spindle waves are normally mediated through the activation of GABA_{A} receptors on thalamocortical neurons. When these receptors are blocked by picrotoxin (20 μM), the spindle waves are transformed into events that resemble absence-like seizures. During normal spindle waves, the IPSPs in thalamocortical cells elicited by activation of GABA_{A} receptors last about 100 msec. When GABA_{A} receptors were blocked, the duration of the IPSPs increased to about 300 msec due to activation of GABA_{B} and the oscillations slowed from 7-14 Hz to 3-4 Hz. Since the intrinsic harmonics of the thalamocortical cells (which oscillate preferentially at ˜3 Hz) match that of the thalamocortical-PGN loop (which also oscillates preferentially at ˜3 Hz), these 3-4 Hz bursts became very strong, and generated a massive synchronized discharge at about 3-4 Hz. In this manner, normal spindle waves in vitro can be ‘perverted’ into absence-like seizure-like events.

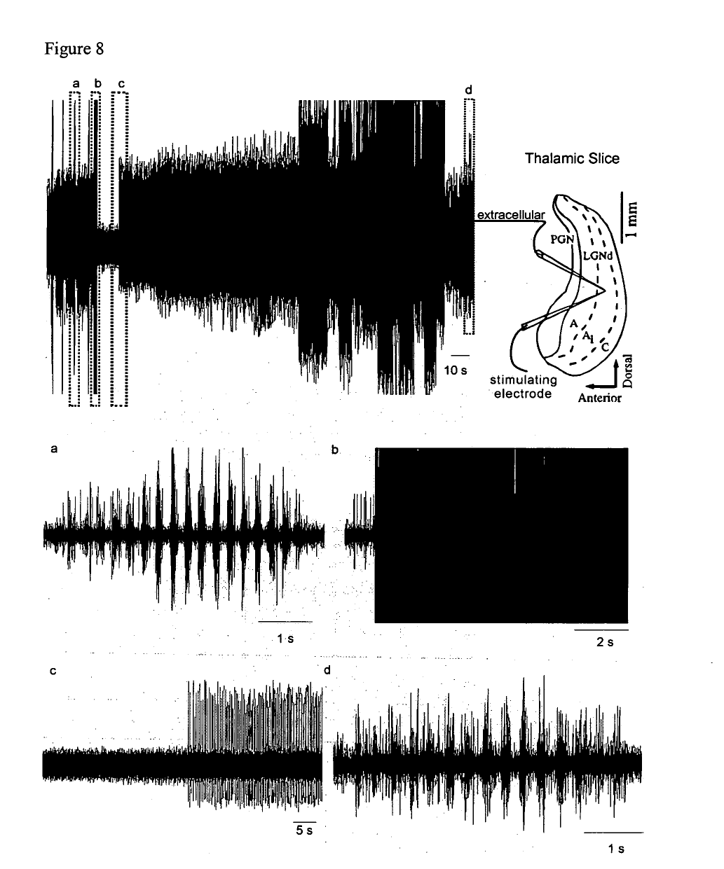

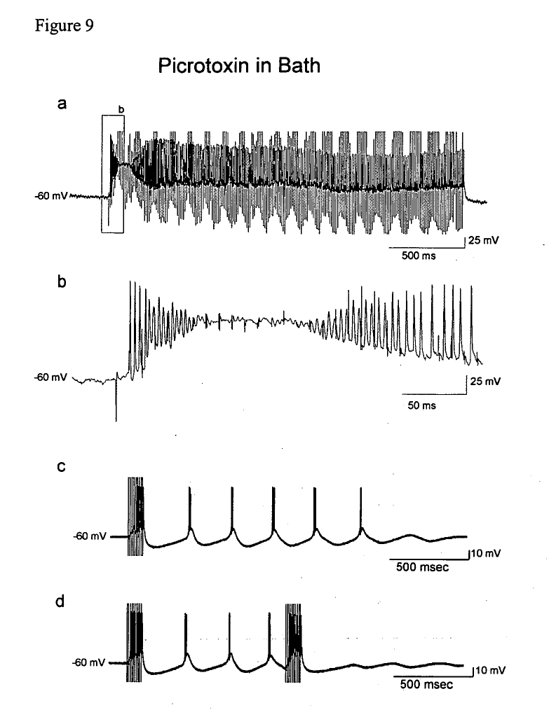

In picrotoxin treated slices, HFS applied to thalamocortical relay neurons eliminated the 3-4 Hz seizure-like activity in 5 slices, as observed using extracellular recording electrodes (see FIG. 8). Intracellular recordings, from picrotoxin treated thalamocortical relay neurons, during HFS (n=5 cells) revealed EPSPs, membrane depolarization, action potential generation, depolarization block, followed by further action potential generation (FIG. 9). The multiple traces shown in FIG. 8 represent time-expanded views of particular portions of the extracellular recording, and are shown as frames (a)-(d). FIG. 8 shows enlargement of an extracellular recording of a spontaneous slowed oscillation (frame (a)); enlargement of an extracellular recording during the stimulation period showing the stimulation artifact (frame (b)); enlargement of portion in (a) showing the return of tonic action potential firing after a period of silence (frame (c)); and reappearance of the slowed oscillations (frame (d)). The initial IPSPs seen in the current clamp in the absence of picrotoxin was not seen, suggesting that the initial IPSPs are mediated by release of GABA and GABA_{A} receptor activation (FIG. 9, frame (a), n=5 cells). In one cell, a brief HFS (100 msec) elicited a slow oscillation (n=1). This is shown in FIG. 9 (frame (b)) which is a time expanded view of the corresponding portion of the trace shown in (a). HFS during the oscillation resulted in EPSPs, membrane depolarization, action potential generation, and abolition of the slowed oscillation.

Conclusion

High frequency stimulation abolished synchronous spontaneous oscillations in the thalamic slice preparation from the ferret. High frequency stimulation seemed to disrupt oscillatory activity by releasing inhibitory and excitatory neurotransmitters. High frequency stimulation disrupts the thalamic circuitry that generates oscillatory activity underlying tremor and absence-like seizure activity. Paradoxically, HFS (excitatory) and surgical lesions of the ventral internal medial thalamus (presumably inhibitory) both suppress tremor. However, HFS-mediated neurotransmitter release and thalamic surgery both disrupt the circuit generating tremor or seizure, albeit by different mechanisms.

### Example 2

The precise mechanism of action of high frequency stimulation (HFS) in the thalamus for the treatment of tremor and epilepsy is unknown. The following experiments were performed to test the hypothesis that HFS results in increased glutamate release and abolishes synchronized oscillations such as spindle waves and 3 Hz absence-like seizure discharges which are generated within the thalamic neural network.

Methods

Direct glutamate measurements were made using a dual enzyme-based electrochemical sensor placed stereotactically in the thalamus of anaesthetized rats. In addition, intracellular electrophysiological recordings were made in the thalamocortical relay neurons and in GABAergic nucleus Reticularis thalami (nRt) neurons in the in vitro slice preparation from the ferret lateral geniculate nucleus. This slice preparation spontaneously generates spindle oscillations and, in the presence of GABA-A antagonists, generates 3 Hz absence seizure-like discharges. Electrical stimulation (100 μSec pulse width; 1 sec-10 min pulse duration; 10-2000 μA amplitude; 100 Hz frequency) was delivered using bipolar stimulating electrode placed within 100 μm of the recording electrodes in both the in vivo and slice preparations. Further, a computational model of the nRt and thalamocortical relay neural network was made and effect of HFS tested within the model.

Results

Thalamic HFS resulted in increased glutamate release in the thalamus which reached a plateau in 2-4 minutes and stayed elevated for the duration of the stimulation period. HFS of thalamocortical relay neurons resulted in the generation of excitatory post-synaptic potentials, membrane depolarization, decrease in the apparent input resistance, and abolition of spontaneous spindle and 3 Hz absence seizure-like oscillations in both thalamocortical relay and nRt neurons during the stimulation period. Similarly, oscillatory behavior within the computational model of the thalamic neural network was also disrupted by simulated HFS.

Conclusion

These results suggest that the mechanism of action of HFS involves the release of glutamate and abolition of spontaneous neural network oscillations. Through this mechanism, HFS may be able to abolish synchronous thalamic network oscillatory activities such as those that generate tremor and seizures.

### Example 3

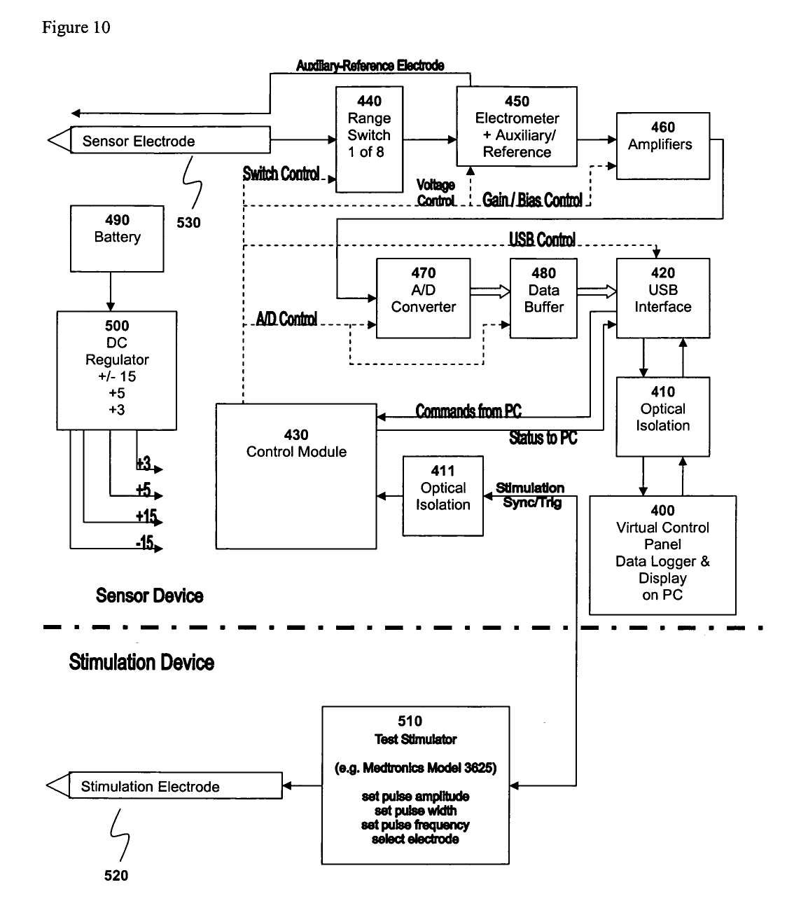

FIG. 10 shows a detailed example of a high frequency brain stimulator useful reducing tremor in an individual. Although the sensor electrodes and the stimulation electrodes are depicted as separate electrodes, they can be combined as a single probe as described above.

Virtual control panel 400 comprises software on a conventional personal computer (PC) that provides control of the sensor electrode. There may be some signaling devices such as LED's (Light Emitting Diodes) on the device to confirm activity and status, but no push buttons, keypads, LCD (Liquid Crystal Display) panels or rotary switches are necessary, but may be included. The functionality of the high frequency stimulation device is entirely controlled from the PC through the Universal Serial Bus (USB) interface 420. The PC will show a graphical image of the CPA device and the various functions of the device (e.g., settings for DC power on-off, electrode potential, electrode selection, gain and amplification, etc.). In addition, the PC can serve as a graphics interface to display data recorded on-line. All data lines and command lines to and from the PC should be passed through optical isolation components to minimize any hazardous current flow from the alternating current (AC) power lines and the patient. The PC can be a conventional personal computer, or can be adapted from a handheld computer such as a PDA or other suitable device.

Optical isolation components 410 and 411 provide electrical isolation between the sensor electrode, the PC and the high frequency stimulation device. An optical isolator converts a pulse of current on the transmit side to a pulse of light. On the receiving side, the pulse of light is converted to a voltage pulse. Control and information is passed from one sub-system to another without physically connecting them with wires and thus hazardous currents being passed to the patient is avoided should an electronic failure occur. It will be understood by one of skill in the art that other modes of electrical isolation may be employed in the high frequency stimulation device of the invention.

USB interface 420 is a high speed serial interface with the PC. External computer devices can be connected to the PC via a simple serial interface cable and the installation procedures are user friendly (plug and play). Digitized recording data and high frequency simulation device status data can pass from the device to the PC for display. Control commands can pass from the PC to the device to establish the proper data collection configuration. Note that the USB interface is optically isolated (410) from the PC to prevent hazardous currents from entering the patient from the AC power lines connected to the PC.

Control module 430 receives commands from the PC (e.g., settings for DC power on-off, electrode potential, electrode selection, gain and amplification, etc.) via USB interface 420. The outputs of this component include “switch control”, “voltage control”, “gain/bias control”, “USB control”, “analog to digital (A/D) control”, and the sensor device “status to PC”. “Switch control” sets the range of current recorded from the sensor electrode via range switch 440. “Voltage control” sets a constant potential (voltage) to the “auxiliary electrode” via the electrometer+auxiliary/reference 450. “Gain/bias control” sets the amplification parameters of amplifier 460. “USB control” monitors and sets data flow through the USB interface 420. “A/D control” monitors and sets the A/D converter 470 and accompanying data buffer 480. “Status to PC” provides system information from the sensor electrode and stimulus information from the stimulation electrode to be continuously monitored by the PC via USB interface 420.

Range switch 440 functions as an electronic switch that permits eight different current ranges to be selected by commands from the PC operating through control module 430. Each setting determines the absolute range of current (e.g., 10 to 100 nanoamperes) that can be measured by the Electrometer 450 at any given time. The sensor electrode 530 makes electrical connection to the device through range switch 440 which, in turn, makes electrical connection to electrometer 450. Although range switch 440 is shown as including eight ranges, range switch 440 can include any number of ranges. For example, it may not be necessary to have a 1 of 8 position range switch 440, but rather a 1 of 3 or 1 of 4.

Electrometer+auxiliary/reference 450 is a two or three-electrode high impedance current measurer and serves to measure changes in current flow through the sensor electrode 530 in tissue or aqueous solutions, via range switch 440. A constant potential (fixed voltage) is also provided to the “auxiliary/reference electrode” connected directly to electrometer 450. The analog output voltage (proportional to the input current to electrometer 450) is fed directly to amplifier 460.

Amplifier 460 comprises circuitry that provides appropriate amplification of the analog output voltage at the output of electrometer+auxiliary/reference 450 circuits. This amplification is necessary to provide suitable voltage levels for the A/D converter 470 circuits. The gain and bias of these amplifier circuits are set as required to maintain signal fidelity by micro-controller 430.

A/D converter 470 serves to convert a voltage from the amplifier circuits of amplifiers 460 (proportional to the input analog current signal to electrometer 450) to a digital signal suitable for data processing. A/D converter 470 is under the control of the micro-controller 430. Digital signals from A/D converter 470 are fed into data buffer 480 for temporary storage. Alternative to what is shown in FIG. 10, A/D converter 470 and amplifier 460 can be a single device which performs both functions of A/D converter 470 and amplifier 460.

Data buffer 480 serves to store and buffer the continuous flow of digital current signals from A/D converter 470 for on-line graphic display on the PC via USB interface 420. Data buffer 480 is under the control of control module 430.

Battery 490 is a direct current (DC) battery that interfaces with DC regulator 500. DC regulator 500 serves as a voltage regulator to deliver power to the electronic units/components comprising the high frequency stimulation device. This form of power supply minimizes any hazardous current from entering the patient from the AC lines supplying power to the PC.

Test stimulator 510 is connected to the stimulation electrode 520 and comprises any pre-existing (e.g., Medtronics 3625 test stimulator) or future electronic stimulation device used for brain stimulation. The signal line “stimulation synchronization/triggering” connecting test stimulator 510 with control module 430, via optical isolator 411, provides communication between the sensor and stimulation components of the device. This communication may be uni- or bi-directional depending on the type of test stimulator employed. A minimal configuration will require uni-directional information of the timing and triggering of stimulation pulses from test stimulator 510 to the PC for the purpose of graphically presenting this information in synchronization with recorded changes in digitized current data from the sensor. This signal will be optically isolated by optical isolation 411 to minimize any hazardous currents flowing into the patient from either of the two electronic devices.

Some of the blocks in the sensor portion of the device of FIG. 10 need not be as complex as shown. Likewise, the gain/bias control circuits may by optionally omitted.

### Example 4

In this example, the hypothesis that HFS to thalamus or subthalamic nucleus (STN) induces astrocytic glutamate release capable of abolishing synchronized neural network oscillations was tested.

Materials and Methods

In vivo Glutamate Measurements in the Rat STN and Thalamus

The in vivo experiments were performed with male or female Sprague Dawley rats weighing an average of 250±55 grams. The rats were housed in plastic and steel cages in a temperature controlled room (21° C.) under a 12 hour light/12 hour dark cycle (light on at 08:00 hr). The rats had ad libitum access to food pellets and water prior to surgery. Before surgery, the rats were anaesthetised with ketamine (100 mg/mL) and xylazine (20 mg/mL). Once anaesthetized, the rats were placed in a Kopf stereotaxic frame in which the skull was secured with a nose clamp, incisor bar and ear bars. Constant body temperature (36.5° C.) was maintained using a heat pad grounded to an external source, and the animal's temperature was measured using a rectal thermometer. A 1.5-2cm incision of the skin was made to expose the cranial landmarks of bregma and lambda. Coordinates for all electrode placements were obtained from the stereotaxic atlas of the rat's brain by Paxinos and Watson. After, a trephine hole was drilled over the left thalamus or STN to allow placement of the recording and stimulating electrodes.

In vivo Electrode Histology

Upon completion of each in vivo experiment, a DC current of 1 mA for 1 s was passed through each recording electrode to mark its position. Rats were then killed by decapitation. Brains were removed, immersed overnight in 10% buffered formalin containing 0.1% potassium ferricyanide and stored in 30% sucrose 10% formalin until sectioning. After fixation, 60-μm coronal sections were cut on a cryostat at 30° C. A Prussian Blue spot resulting from the redox reaction of ferricyanide marked the stimulation site. The placements of stimulating and recording electrodes were determined under a light microscope and recorded on representative coronal diagrams.

Glutamate Electrochemistry

Glutamate biosensors (Pinnacle Technology Inc., Lawrence, Kans.) were manufactured as described by Hu et al. J Neurochem. 1997 68:1745-1752. In brief, the sensor was made using lengths of Teflon-coated platinum iridium (7%) wire (Pt—Ir, 0.25 o.d., Medwire, Mount Vernon, N.Y.). A 0.05 mm Ag wire was wrapped on the Teflon coated Pt—Ir electrode and anodized to create an Ag/AgCl reference counter electrode. The sensing cavity was formed by stripping the Teflon coating from one end, revealing the bare Pt—Ir electrode (0.35 mm and 1.0 mm lengths). An interferent screening inner-membrane was fabricated on the bare Pt—Ir electrode. An enzyme layer was formed over the inner-membrane by co-immobilizing glutamate oxidase and ascorbate oxidase with glutaraldehyde and bovine serum albumin (BSA). Glutamate biosensors were tested in 0.1 M phosphate-buffered saline (PBS; 7.4) for a minimum glutamate sensitivity of 300 pA/uM and for insensitivity to ascorbate (response to 250 uM ascorbate less than 0.5 nA). Sensors that did not meet these criteria were rejected. Sensor lengths were manufactured for use with brain slices, with the electrode shaft at ˜15 mm with a sensing region of ˜350 um.

In vitro Thalamic Slice Preparation

For the preparation of slices, 3-4 month old male or female ferrets (muftela putoriouf furo; Marshall Farms; North Rose, N.Y.) were deeply anesthetized with sodium pentobarbital (30-40 mg/kg) and killed by decapitation. The forebrain was rapidly removed, and the hemispheres were separated with a midline incision. Four hundred micron thick slices were cut using a vibratome (Ted Pella, Inc.) in the sagittal plane. During preparation of slices, the tissue was placed in a solution (5° C.) in which NaCl was replaced with sucrose while maintaining an osmolarity of 307 mOsm to increase tissue viability. Slices were placed in an interface style recording chamber (Fine Sciences Tools) maintained at 34±1° C. and allowed at least two hours to recover. The bath was perfused with artificial cerebrospinal fluid (aCSF) which contained (in mM): NaCl, 126; KCl, 2.5; MgSO₄, 1.2; NaH₂PO₄, 1.25; CaCl₂, 2; NaHCO₃, 26; dextrose, 10 and was aerated with 95% O₂, 5% CO₂ to a final pH of 7.4. For the first 20 minutes of perfusion of the thalamic slices, the bathing medium contained an equal mixture of aCSF and the sucrose-substituted solution.

Electrophysiology

Intracellular recording electrodes were formed on a Sutter Instruments P-2000 laser micropipette puller from medium-walled glass (WPI, 1B100F). Micro-pipettes were filled with 2 M K-acetate. Only those neurons exhibiting a stable resting membrane potential of at least −60 mV and electrophysiological properties were included for analysis. Electrical stimulation was achieved through the placement of a concentric stimulating electrode and delivering stimulation (100 μsec duration; 10-500 μA amplitude; 100 Hz frequency). Mean values are given±SEM. The data was analyzed using Chart (eDaq) on a Pentium style computer and figures were drawn using CorelDRAW (Corel).

Primary Astrocyte Culture

Astrocyte cultures were prepared from the cortices of neonatal rats (1-3 day old) using the Worthington Papain Dissociation System (Worthington Biochemical Corporation, Lakewood, N.J.). Briefly, cortices of neonatal rats were dissected, treated with papain (20 U/ml), dissociated by trituration and plated in 75 cm^{2 flasks in Dulbecco's modified Eagle's medium supplemented with} 10% charcoal-stripped FBS and 1% penicillin/streptomycin (100 U/ml penicillin, 100 μg/ml streptomycin). Cells were fed twice weekly until they reached confluence (Day 10-12 in vitro) at which point they were mechanically shaken for 1 hr on an orbital shaker to remove any remaining oligodendrocytes and microglia. Subsequently, cultures were treated with trypsin for 30 mins at 37° C., placed in an eppendorf tube and centrifuged at 100 g for 5 minutes. The cells were washed 2× in PBS prior to inserting the stimulating and glutamate recording electrodes into the cell pellet containing ˜2.0×10⁶ cells.

Immunocytochemistry

Astrocytes on coverslips were washed three times in PBS and then fixed in 4% paraformaldehyde for 10 minutes at room temperature. After rinsing again in PBS, coverslips were blocked in 10% normal goat serum for 30 mins followed by overnight incubation at 4° C. with primary antibody (mouse anti-rat GFAP, 1:500, GA5 clone, Sigma). The following day, slides were washed in PBS and secondary antibody was applied for 2 hours (goat anti-mouse Alexa Fluor 488 or 555, 1:250, Molecular Probes). After a final wash, cells were post-fixed in acid-alcohol (95% ethanol, 5% glacial acetic acid) for 10 mins, rinsed and mounted with VectaShield (Vector Laboratories), examined with an Olympus fluorescence microscope, and images were captured with a Q-Fire cooled camera.

Results

Effect of HFS on Glutamate Release in the Thalamus and STN in vivo

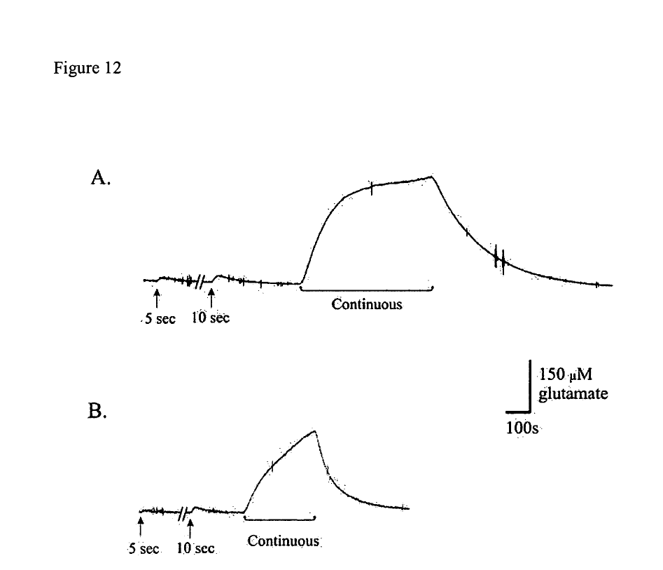

To test the hypothesis that glutamate was the neurotransmitter released during HFS, the extracellular glutamate concentration was measured using a dual enzyme-based electrochemical sensor in the STN and the ventrolateral (VL) thalamus of the rat in vivo. The concentric bipolar stimulating electrode and the glutamate sensor electrode were positioned within ˜200 μm of each other in the STN and VL thalamus in the anaesthetized rat placed in a Kopf stereotactic frame. HFS of the STN (100 Hz, 100 μs pulse width, 300 μA) resulted in an increase in extracellular glutamate in the STN (FIG. 12A; n=13). A similar increase in glutamate level was measured in the thalamus when HFS was delivered to the VL thalamus (FIG. 12B; n=10). Additionally, continuous stimulation of the STN or VL thalamus resulted in an immediate elevation of the glutamate level that remained elevated for the duration of the stimulation. Upon cessation of stimulation, the glutamate level slowly returned to pre-stimulation baseline. The correct placements of stimulating and recording electrodes in the STN or VL thalamus were confirmed under a light microscope in the sectioned rat brains.

HFS Effects on Synchronized Oscillations

To test the functional effects of HFS-mediated glutamate release, simultaneous intracellular and extracellular recordings were made in the ferret lateral geniculate nucleus (LGN) in vitro slice preparation, which generates spontaneous spindle waves (FIG. 7; n=21). Spindle activity was recorded from a population of neurons (extracellular electrode), and the electrophysiological activity associated with the spindle activity was recorded from single neurons within that population. The intracellular recording revealed synchronized Ca²⁺ bursts (FIG. 7a, lower trace and FIG. 7e) concurrently with the population spindles (FIG. 7a, upper trace). HFS was applied by a stimulating electrode positioned within ˜100 μm of the intracellular and extracellular recording electrodes. It was not possible to observe the effect of HFS on spindle activity during HFS due to the stimulation artifact in the extracellular trace (FIG. 7b, upper trace). However, intracellularly, the stimulation artifact did not prevent the observation of an initial IPSP followed by a prolonged EPSP, membrane depolarization, action potential generation, depolarization block and further action potential generation (FIG. 7f; n=21). In the immediate post-stimulation period, neuronal activity was absent (FIG. 7c). The activity returned gradually in approximately 10-30 seconds while spindling returned in 30-60 seconds (FIG. 7d; n=21), indicating that the neurons were not lesioned or damaged (FIG. 7d).

When GABA_{A} receptors were blocked by picrotoxin (20 μM), the spindle waves were transformed into events that resemble absence-seizure-like activity (FIG. 8; n=5 slices). In the picrotoxin-treated slices, HFS applied to thalamocortical relay neurons eliminated the 3-4 Hz seizure-like activity in 5 slices, as observed using extracellular recording electrodes (FIG. 8). Thus, HFS abolished both normal spontaneous synchronized oscillations, such as spindle waves, and abnormal oscillations such as, absence-seizure like 3 Hz oscillations. FIG. 8(a) shows an enlargement of an extracellular recording of a spontaneous slowed oscillation. FIG. 8(b) shows an enlargement of an extracellular recording during the stimulation period showing the stimulation artifact. FIG. 8(c) shows an enlargement of a portion showing the return of tonic action potential firing after a period of silence. FIG. 8(d) shows the reappearance of the slowed oscillations.

Amperometry/Glutamate Electrode

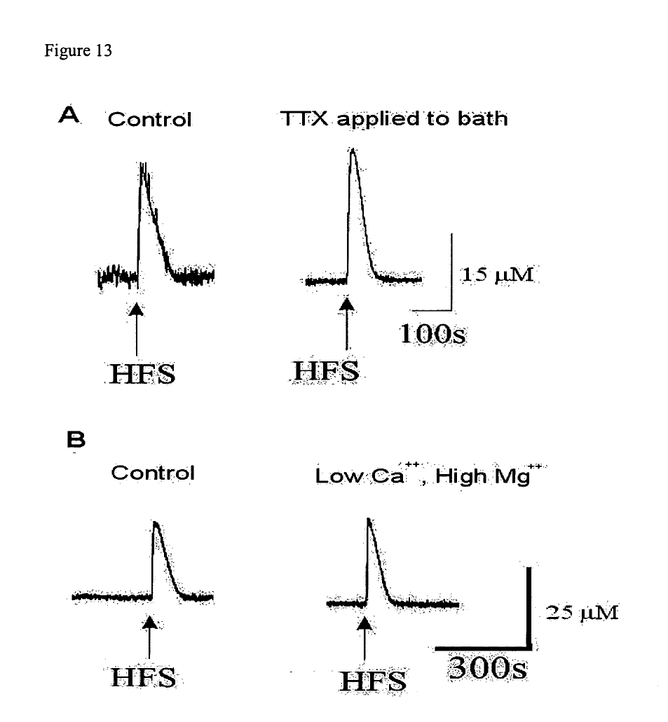

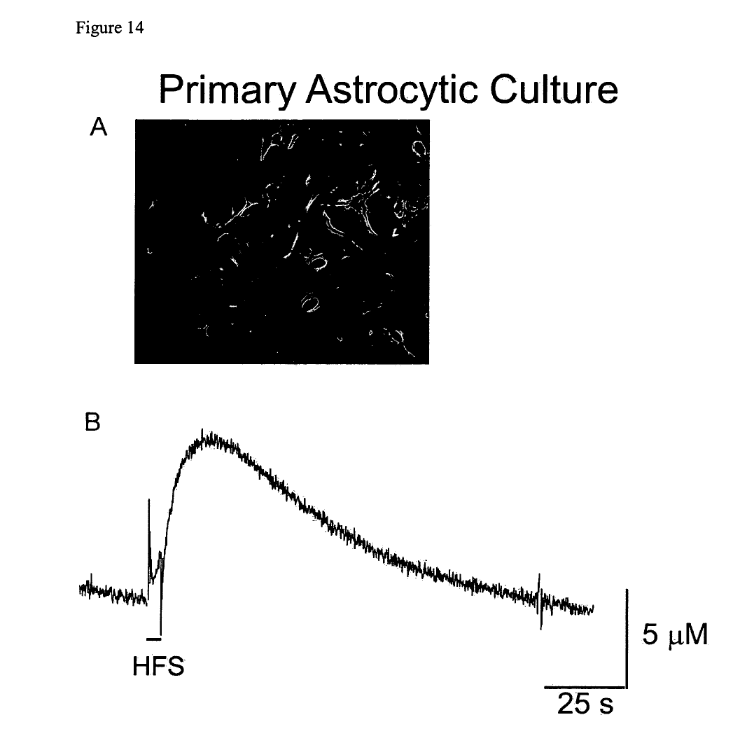

To confirm that glutamate was in fact responsible for the functional consequences of HFS, the extracellular glutamate concentration was also measured in the ferret thalamic slices in vitro using a glutamate sensor. The stimulating electrode and the glutamate sensor electrode were positioned within ˜100 μm of each other and placed in the A1 lamina of the LGN. During HFS (100 Hz, 100 μs pulse width, 300 μA), an increase in extracellular glutamate levels was measured in the control solution (FIG. 13, control; n=10) that was similar in characteristic to the increase measured in the rat in vivo (FIG. 12). To block the neuronal release of neurotransmitters, we used the Na+ channel blocker tetrodotoxin (FIG. 13A, TTX) or a high Mg++, low Ca++ solution (FIG. 14B). However, these classic neuronal exocytosis inhibitors failed to block the glutamate release induced by HFS.

Amperometry/Glutamate Electrode in the Primary Astrocytic Cultures

To test whether the glutamate released from HFS was of astrocytic origin, we utilized a primary astrocytic culture that was >98% pure as determined by glial fibrillary acidic protein (GFAP), a marker for astrocytes (FIG. 14A). HFS (10s duration, 100 Hz, 100 μs pulse width, 300 μA) of the purified astrocytes with the stimulating electrode and the glutamate sensor electrode positioned within ˜100 μm of each other, resulted in an increase in extracellular glutamate as measured by an enzyme-linked glutamate sensor. The glutamate level decreased to baseline upon cessation of stimulation. Of note, the HFS evoked glutamate release profile was similar to the glutamate release profile observed in the rat in vivo and in the ferret thalamic slices in vitro.

### CONCLUSION

These results suggest that HFS of the thalamus or STN leads to glutamate release from astrocytes that is insensitive to classic neuronal exocytosis inhibitors. HFS leads to astrocytic glutamate release and is able to abolish both normal spindle oscillations and abnormal 3 Hz absence-seizure-like oscillations. Thus, astrocytic glutamate release may be an important mechanism by which DBS is able to block abnormal neural network oscillations such as those that may be generated in tremor and seizures.

Other embodiments will be evident to those of skill in the art. It should be understood that the foregoing detailed description is provided for clarity only and is merely exemplary. The spirit and scope of the present invention are not limited to the above examples. All patents and publications cited herein are incorporated by reference in their entirety.

1. A method of providing high frequency stimulation to the brain of an individual in response to detection of an epileptic oscillation comprising the steps of:
(a) providing a high frequency stimulation system comprising a control module, a detection module containing electronic circuitry capable of detecting an epileptic oscillation, and a high frequency stimulation module under control of said control module;
(b) monitoring for the presence of said epileptic oscillation using said detection module;
(c) sending a signal indicative thereof to said control module if said epileptic oscillation is present; and
(d) said high frequency stimulation module generating a high frequency stimulation to the brain of said individual pursuant to a signal from said control module.

2. The method of claim 1, wherein said high frequency stimulation module comprises a stimulation electrode.

3. The method of claim 2, wherein said stimulation electrode is placed in the thalamus of said individual.

4. The method of claim 1, wherein said detection module comprises a sensor.

5. The method of claim 4, wherein said sensor is placed in the thalamus of said individual.

6. The method of claim 1, wherein said high frequency electrical stimulation signal is between about 10 and 1000 μA in amplitude, has a pulse width of between about 10-500 μs, has a frequency of between about 10-500 Hz, has an output voltage of between about 1-20 V and has a duration of between about 1 second and 12 hours.

7. The method of claim 1, wherein said epileptic oscillation is a thalamic oscillation of 3-6 Hz.

8. The method of claim 1, wherein said epileptic oscillation is a decrease in thalamic oscillation from 7-14 Hz.

9. The method of claim 1, wherein said high frequency stimulation system further comprises a chemical delivery module operably connected to said control module, wherein said method further comprises the step of said control module sending a signal to said chemical delivery module if said epileptic oscillation is present, wherein said chemical delivery module delivers a neuroactive compound to the brain, or portion thereof, of said individual.

10. The method of claim 7, wherein said neuroactive compound is selected from the group consisting of neurotransmitter, neuropeptide, neurochemical, receptor agonist, receptor antagonist, ion channel blocker, and ion channel activator.

11. A method of reducing tremor in an individual comprising:
(a) placing a sensor in or on the brain of said individual, said sensor capable of measuring an epileptic oscillation in said individual, and said sensor being operably connected to a control module;
(b) placing a stimulation electrode in or on the brain of said individual, wherein said stimulation electrode is operably connected to said control module;
(c) measuring thalamic oscillation from said individual;
(d) detecting an epileptic oscillation in said thalamic oscillation, wherein if said epileptic oscillation is detected, a signal indicative thereof is sent to said control module; and
(e) said high frequency stimulating module generating a high frequency stimulation signal pursuant to a signal from said control module, and applying said high frequency stimulation signal to said stimulation electrode, whereby said tremor is reduced.

12. The method of claim 11, wherein said stimulation electrode is placed in the thalamus of said individual.

13. The method of claim 11, wherein said sensor is placed in the thalamus of said individual.

14. The method of claim 11, wherein said high frequency electrical stimulation signal is between about 10 and 1000 μA in amplitude, has a pulse width of between about 10-500 μs, has a frequency of between about 10-500 Hz, has an output voltage of between about 1-20 V and has a duration of between about 1 second and 12 hours.

15. The method of claim 11, wherein said epileptic oscillation is a thalamic oscillation of 3-6 Hz.

16. The method of claim 11, wherein said epileptic oscillation is a decrease in thalamic oscillation from 7-14 Hz.

17. The method of claim 11, wherein said high frequency stimulation system further comprises a chemical delivery module operably connected to said control module, wherein said method further comprises the step of said control module sending a signal to said chemical delivery module if said epileptic oscillation is present, wherein said chemical delivery module delivers a neuroactive compound to the brain, or portion thereof, of said individual.

18. The method of claim 17, wherein said neuroactive compound is selected from the group consisting of neurotransmitter, neuropeptide, neurochemical, receptor agonist, receptor antagonist, ion channel blocker, and ion channel activator.

19. The method of claim 11, wherein said thalamic oscillation is measured by EEG.

20. A system for treating tremor in an individual comprising:
(a) a control module including electronic circuitry;
(b) a high frequency stimulating module operably connected to said control module;
(c) a sensor operatively connected to said control module capable of recording a thalamic oscillation signal from said individual, said sensor adapted to be placed in or on the brain of said individual;
(d) a stimulating electrode operatively connected to said high frequency stimulation module, said stimulating electrode adapted to be placed in or on the brain of said individual;
wherein said electronic circuitry of said control module is adapted to determine if an epileptic oscillation is present in said thalamic oscillation, and to send a signal to said high frequency stimulating module if said epileptic oscillation is present, wherein said high frequency stimulation module generates a high frequency stimulation which is delivered to said stimulating electrode.
